(19) United States
(12) Patent Application Publication
(10) Pub. No.: US 2011/0027875 A1
(43) Pub. Date: Feb. 3, 2011

(54) Inexpensive, Vertical, Production Photobioteactor

(76) Inventor: Paul Cathcart, Sacramento, CA (US)

Correspondence Address: GEORGE PHILIP WHITE;Patent Agent, 533 HAVANA AVE, LONG BEACH, CA, 90814, US

(21) Appl. No.: 12/834,556

(22) Filed: Jul. 12, 2010

Related U.S. Application Data
(60) Provisional application No. 61/225,527, filed on Jul. 14, 2009.

(51) Int. Cl. C12M 1/04; C12M 1/00

(52) U.S. Cl. 435/292.1

(57) ABSTRACT

A production photobioreactor for culturing microalgae has a primarily vertical orientation of parallel transparent columns or tubes connected in a closed loop serpentine fashion. Generally no pump is required. The flow is maintained by gas diffusion in alternate columns (up columns) having a rate and volume as to provide an air lift with sufficient rate and volume to cause fluid to rise and cascade into adjacent “down” columns. Adequate mixing in the down columns is engendered by a bubble flow at a rate less than that of the up columns.

### RELATED APPLICATIONS

This applications claims priority from U.S. provisional utility application 61/225,527 filed Jul. 14, 2009.

### FIELD

This invention is in the field of closed photobioreactors intended for growing microalgae, or the like.

### BACKGROUND

Algae are rich in many interesting and important compounds and there is a great potential for using algae as human food, food additives, nutritional supplements, animal feed, aquaculture feed, feedstock for chemical manufacturing, feedstock for bio-fuels and for various other purposes.

Closed photobioreactors may be used to grow algae in an environment with controlled amounts of nutrients and CO₂ and light By being closed systems, rather than an open pond or pool, undesired material and organisms can be minimized more effectively. In closed systems culture parameters such as pH, nutrient levels, temperature, Co2, light, etc can be controlled thus allowing for precise culture conditions. Applying different stresses to the algal cells can produce a desired result such as deprivation nitrogen, which results in lipid synthesis. Or high irradiation combined with nitrogen deprivation for the synthesis of carotenoids. Also, forces tending to mix the media can be applied to more predictable effect.

While many diverse designs have been proposed, studied and constructed, the problem of “scale up” from lab results to production systems remains. The constraints on the problem are significant: support a large mono-culture (generally requires a rapid recirculation), limited shear applied to cells, mixing to insure that cells receive light in the proper degree and timing, inexpensive, and quick to tear-down and clean to start a new culture when an existing culture needs to be replaced.

There have been some notable failures in approaches to scaling up a lab bioreactor into successful, cost effective, production facilities. The literature includes statements that this field is “as much an art as a science” and it has also been noted, “it seems that every researcher has their own bioreactor design”. Most known designs fall into the categories of being essentially horizontal or essentially vertical. Systems constructed of serpentine connected rows of horizontal tubes are often called “tube reactors”. They have an advantage of a single culture recirculating through conduits in an orientation where all areas are affected similarly by gravity as the media is pumped around the loop.

However, these systems have notable issues. Tube diameters are narrow to allow for high surface area to volume ratios. This means there is a high level of friction to overcome to circulate the culture. Pumping at the pressures needed to circulate can produce sheer stresses, which can result rupturing of the cell wall and subsequent cell death. Another point is the removal of O2 gasses that are the product of photosynthesis. High O2 levels inhibit algal growth and therefore should be removed. In the horizontal sections of tubing the O2 is not removed and builds up concentration.

These systems typically use vertical degassing columns to remove the O2. Because of the narrow diameter of the tubes, the tube length, and the propensity of cell death from shear stress, turbulent flow is normally not achieved. Laminar flow in tubular systems can result in over exposure to light of the algal cells traveling near the tube wall. This can cause photo inhibition. The algal cells traveling near the center of the tube will be shaded and may not receive sufficient light for optimum growth. Also, tubes can get clogged up and coated with algae, which can lead to decreased light transmission rates through now not so clear tubes, slowing the algal growth rates, and requiring more labor to clean the system plus the risk of contaminating the system with other species. Long horizontal tubes have difficulty addressing this issue in a cost-effective manner.

Vertical systems have an advantage in being able to use an upward flow of rising bubbles to accomplish a relatively gentle (compared to a pump) but turbulent mixing. With a greater degree of injected gas an “airlift” tending to cause a circulation of media can also be achieved. One approach, a split cylinder airlift vertical column reactor, can achieve turbulent mixing and a good recalculating flow rate without a pump but is limited in size to a few meters in height and a few tenths of a meter in diameter.

### SUMMARY

The present teaching is of apparatus and methods related to a vertical, serpentine, recirculating photobioreactor, versions of-which address many of the above deficiencies.

In some versions, a series of adjacent, parallel, vertical columns are interconnected in a serpentine, closed loop, fashion. To accomplish this “plumbing” 180-degree fittings or couplers can be alternating used to connect adjacent tops and then adjacent bottoms of the vertical columns. Nominally, they these are alternating “up” columns and “down” columns.

It is consistent with this invention that the flow of media is substantially engendered by bubble or airlift gas flows in the up columns of sufficient force and volume as to move media upward in the up columns, providing enough momentum to cause a corresponding downward flow in the alternating located down columns. Embodiments generally have both the up and down columns transparent and exposed to light to facilitate photosynthesis over a maximized surface area. The bubbles in the up columns perform both the functions of engendering system-wide recirculation and that of mixing cells in the up columns. The down columns may not benefit to same degree from the mixing effect of the up column's bubbles. Although the media flow will cascade and continue downward, the bubbles generally do not.

Without further mechanism the above design might not be successful in that the cells near the surface in the down columns could be overexposed and die often sticking to the surface of the column and then blocking light. A solution taught herein is to include bubble insertion at the bottom of the down columns. The rate and volume could be great enough to engender adequate down-column mixing but low enough compared to the up-column gas insertion that the desired rate of recirculation is also obtained.

In order to avoid or minimize the need for pumping, the connection from the far-end column in a serpentine back to the starting column can be accomplished in some versions by configuring the system other than linear, as seem from above. For example, by offsetting the column-to-column upper and lower connections by 6°, a 60-column system would constitute a circle as seen from above, placing the last column immediately adjacent to the first column.

The relatively low pressures present in the columns allow them to be, in some versions, composed of an elongated Low Density Poly Ethylene extrusion. Essentially, the columns can be plastic bags.

This summary is intended to introduce aspects of the present invention whose metes and bounds are delineated in the claims.

### OVERVIEW OF DRAWINGS

FIG. 1. Schematic view of a version of an 16-column bioreactor;

FIG. 2. Perspective view of a bioreactor similar to that of FIG. 1 but shows a 20-column unit;

FIG. 5. Expanded view of one segment of a bioreactor;

FIG. 6A Further expanded view showing only the lower U coupling, diffusers, and gas manifold.

FIG. 6B shows an exploded version of FIG. 6A.

FIG. 9 perspective of lower connections of columns, U coupler, and manifold

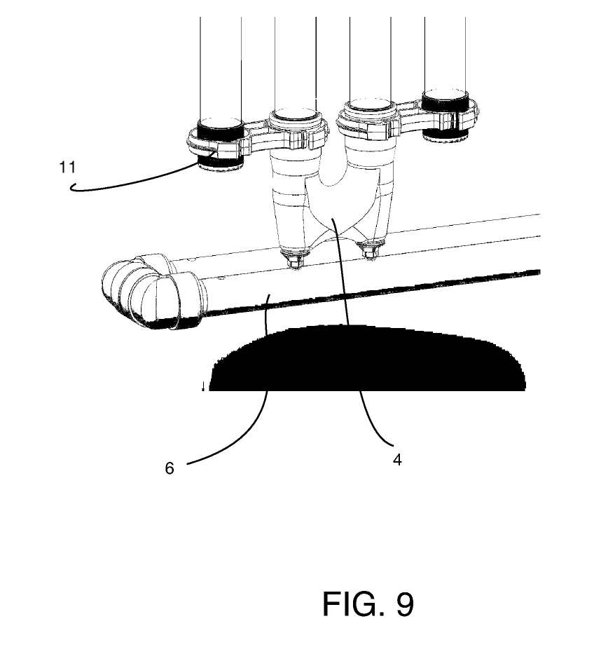

FIG. 11 perspective view of lower U coupler showing lower part of diffusers

FIG. 12 A perspective view of alternate upper U coupler with a single gas-out auxiliary port.

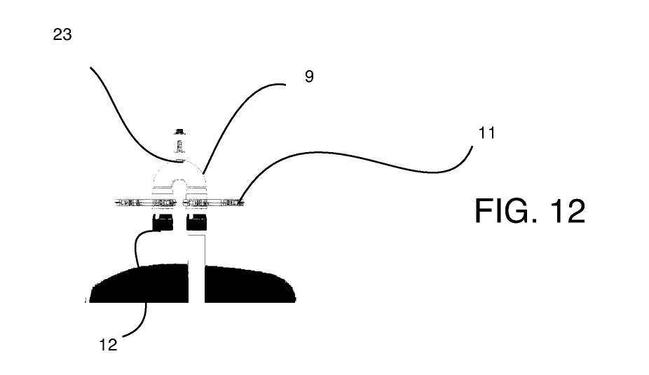

FIG. 13 A perspective drawing of a 50-column unit

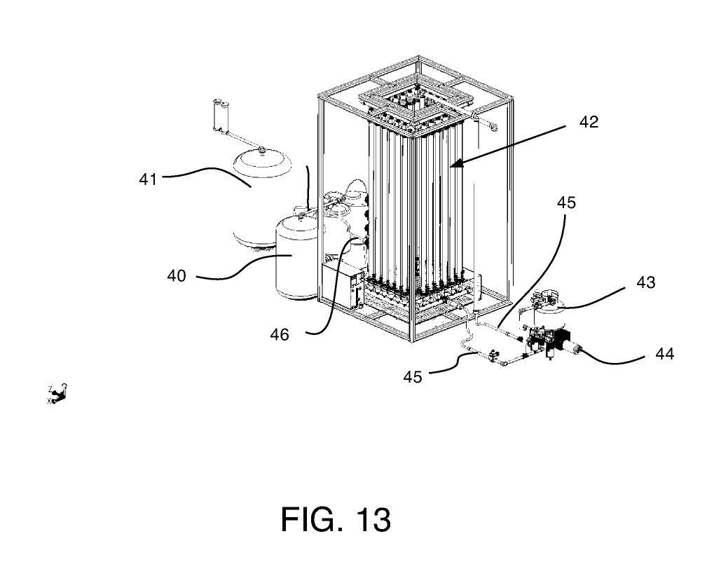

### DETAILED DESCRIPTION

The descriptions of specific versions of equipment and methods consistent with the teachings herein are presented to better explain how to make and use items using the principles of this invention.

### First Presented Version

A photobioreactor is constructed primarily from vertical, transparent, columns of a flexible LDP bag material connected in a serpentine fashion by 180-degree PVC U-fittings or couplers at the top and at the bottom.

Basic Structure

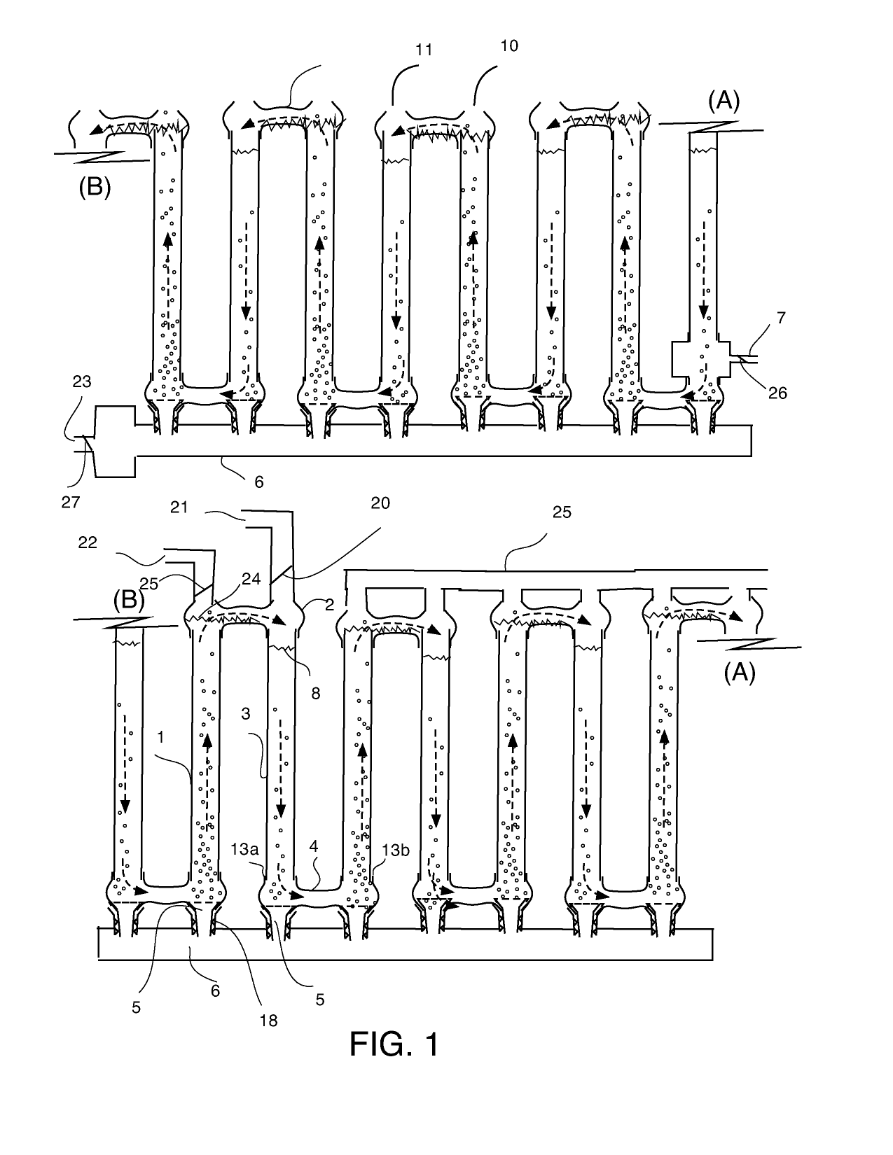

As seen in FIG. 1, the basic stricture consists of an array of adjacent, parallel vertical columns. They are alternating “up” columns 1 and “down” columns 3. The top of each up column is connected by an upper U-coupling 2 to the next down column. The U-couplers have two primary ports 13b 13a facing in a common direction and connected by a fluid conduit. Correspondingly, the bottom of each down columns is coupled by a lower U-coupled to an adjacent up columns. In the version shown in drawings 1 and 5, the upper and lower U-couples are constructed a common component.

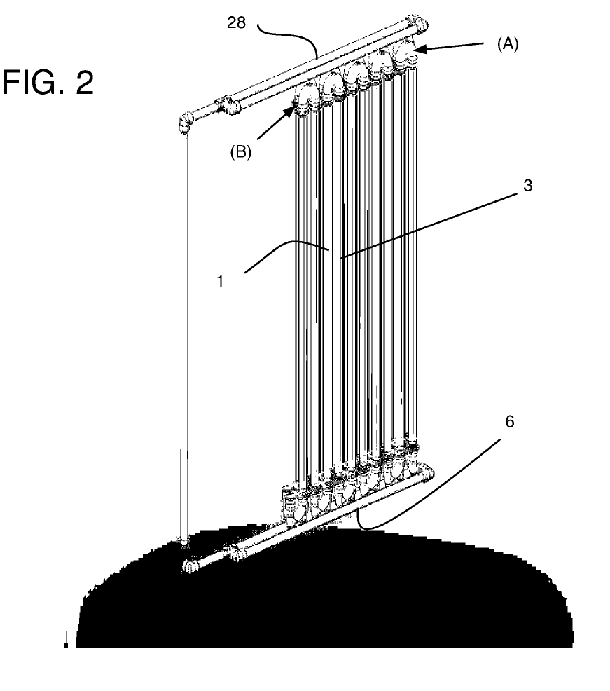

In order to have recirculating system with uniform characteristics throughout, this version has its beginning adjacent to its end. FIG. 1 shows two rows of eight columns each. In fact, this is a schematic representation of a single closed 16 column recirculating system. The two rows are physically located back-to-back as seen in FIGS. 2 & 3. The two point A's are touching each other as are the two point B's. The couplers at those points are oriented about 45-degree offset from the line in which their adjacent couplers lie.

The lower U-couplers have gas diffusers 5 in each of their auxiliary ports 19a 19b immediately opposite the two primary ports 13a 13b. The diffusers are oriented to supply bubbles into their respective columns. The gas diffusers are connected to a gas manifold 6 running perpendicular to the orientation of the columns.

There are three input ports and at least one output port in the version shown in FIG. 1. One input port 22 is intended to be an entry point for innoculum into the system. Another port is the media/nutrient port 21. The third port is a gas input port 23 on the gas manifold 6. These, as well as the output port for harvesting 7 have valves 20 25 26 27 for controlling their use. Not shown are standard structures involved in pre-mixing, pre-filtering and pre-sterilizing input material as required. Also not shown are devices for containing and processing harvested material. An upper manifold, shown in one of the rows of FIG. 1 and both rows in FIG. 2 may be present in some versions and installations to take out-gas from the upper U-coupler auxiliary ports and channel, test or process it in some desired manner.

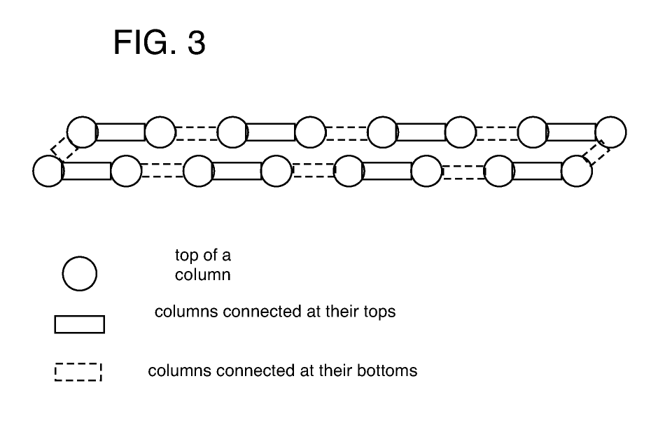

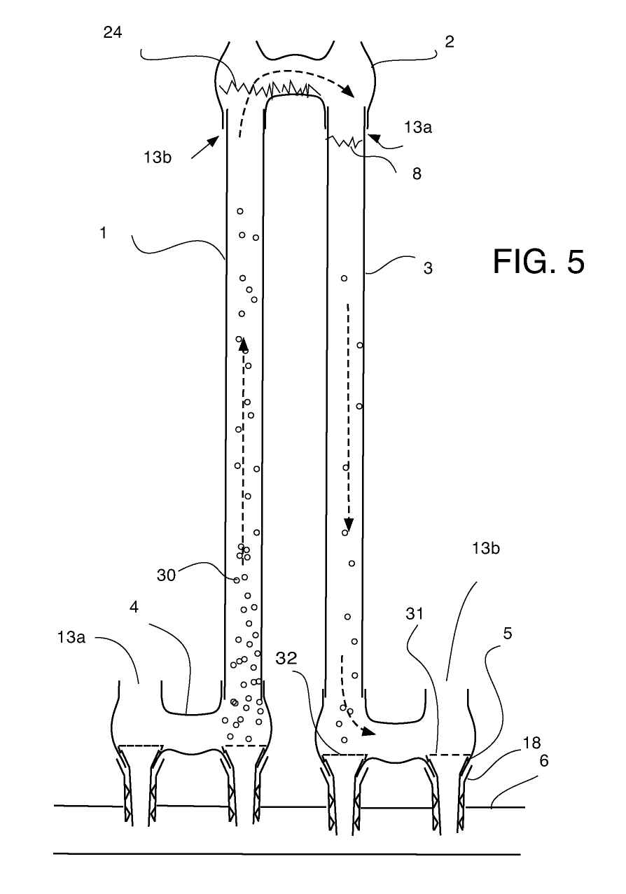

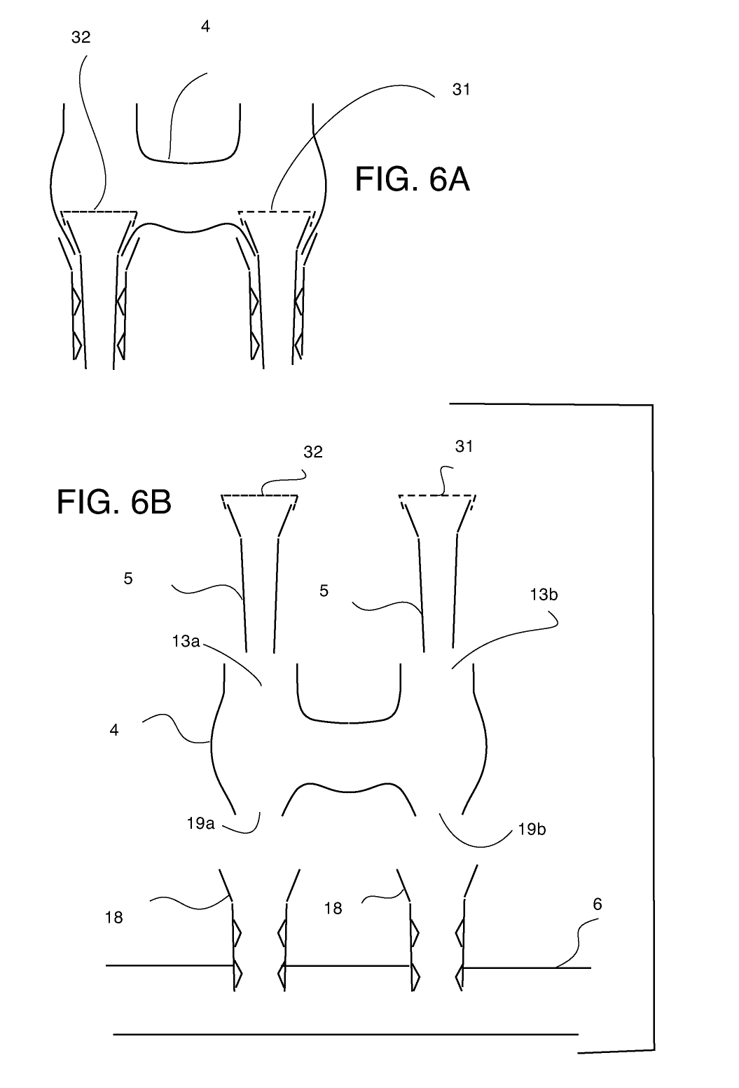

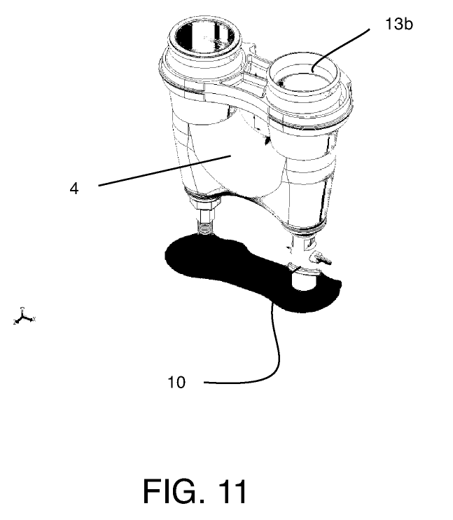

As seen more clearly in FIG. 5 and FIG. 6, the diffuser's bodies are shaped somewhat like funnels with the straight tube connected to the gas manifold and the open conical area facing up into the U-coupler and the bottoms of both up and of down columns. The up and the down diffuser bodies may be composed of a common part. FIGS. 1 and 5 show, however, a greater volume of bubbles 30 in the up columns than in the down columns. This is due to structural or adjustment difference in up diffusers and down diffusers. As shown in FIGS. 5 and 6, identical diffuser bodies are capped by distinct elastomeric membranes. In one version that has been constructed and tested, both membranes have 300 small holes, with the up diffuser's membranes 31 having slightly larger holes than those in the down diffuser's membrane. Alternate versions might have other means of regulating the two bubble flow rates (up/down), such as via a different point of fixed constriction or via adjustable control valves. FIG. 11 shows a lower sub assembly with one of the valve bodies 10 having an adjustable flow.

Macro Structure Configuration

Units corresponding to FIG. 1-6 have been constructed using 2″ diameter columns that are 2 meters tall. In this version the columns in each continuous row are evenly spaced on 6″ centers. There are many alternate configurations that bring the “end” column back adjacent to the “start” column. They may be in a simple circle or in back-to-back concentric circles. Other macro configurations that are possible include an “E” shape (as seem from above). Due to the relatively lightweight of the construction, bioreactors consistent with these teachings may often be supported primarily by cables from above connected to an upper manifold. The cables might be hung from a lightweight rack structure or attached to the ceiling of a greenhouse, for example.

Also, common with other bioprocess units, computerized control systems are generally used for efficiently and safety of operation. These structures and operations are well known to those skilled in the art.

Operation

There are two aspects of “operation”. One is how it operates and the other is how to operate it.

How it operates:

Some desirable attributes of a production photobioreactor include:
a) Significant total volume of a single culture minimizing the number of input and output ports required per volume of generating ability.
b) Continuous, steady, recirculating flow. Fast enough to ensure a monoculture but not forceful enough at any point to present a significant portion of the culture to excessive shear forces.
c) Turbulent mixing effective at rotating cells between outer areas exposed to light and inner areas shaded by other cells. Too much light is bad and too much dark is bad.
d) Have a very high percent of the flow path be transparent and exposable to a light source.
e) Readily sterilizable and inexpensive and quick to take down, clean and restart.
f) Provision for gas injection in an evenly distributed fashion.
g) Provision for out-gas extraction in a distributed fashion.

A steady flow over a large volume without undue cell stress at any point is achieved, generally by the eliminating or minimizing the role of pumps. Every other column is an up column 3 with a diffuser 5 generating a flow of air bubbles sufficient to displace an adequate volume of media/culture. An adequate level is a water level 24 at the top of a column that is high enough to enter the upper U-coupler and cascade through the coupler into the adjacent down tube. The adjacent down tube, not having the same bubble activity will have a lower water level 8. And so on, circulating through column-to-column. This is the basic mechanism of flow generation through the system.

Besides pushing the level high enough to engender the primary flow, in the manner of an air lift reactor, the gas from each up diffuser also causes a turbulent flow in its respective up column. Turbulent flow is effective at rotating cells between the central portions and surface portions of the media. This mixing promotes efficient photosynthesis without under- or over-exposing cells to the light.

As described so far in this operation section, a bioreactor would not likely be successful. Mixing would be deficient in the down columns causing cells with excess 220 surface time to be over-exposed while the cells with excess interior time would be under-exposed. Approaches to solve this include: darkening the down columns, engendering mixing in the down columns by vibrations, magnetically operated stirrers or other stirring mechanisms. As shown in FIGS. 1, and 3 another approach is to bubble the down columns but to a lesser extent than the up columns. One might 225 think of the system as alternating air lift columns and bubble columns. In practice, with 2″ diameter, 2 meter tall columns one way to determine adequate bubble flows would be to (1) determine bubbling needed just for mixing, (2) determine net gas flow for desired forward flow rate. (3) Use first figure for down column flow rate and sum of both figures to use for up column gas rate.

In the versions shown in FIG. 1-6, both types of diffusers are of identical construction but with different diffusing membranes. Each membrane is of the same material and each has the same 300-hole pattern. The up diffuser has larger holes due to the same 300-needle die being pressed into the up diffuser under a higher pressure.

As steps, the flow progresses:

In a vertical serpentine bioreactor partially filled with a flow-able, pourable media these actions occur (in no specific time ordering)
i. Gas bubbles rise up from a diffuser into one or more columns. The intensity, volume or rate of bubble is such as to raise the fluid level from not completely full to overflowing those columns into adjacent columns;
ii. The gas bubbles of action (i) engender turbulence in their respective columns;
iii. Gas is bubbled into the mentioned adjacent columns but at a rate less than that of action 1;
iv. Optionally the fluid from the last column in the serpentine sequence is introduced into the first column in the serpentine constituting recirculation.

How to work it:

Steps to set up a bioreactor for an N-column bioreactor (not necessarily in order):

1) Provide
N U couplers;
N×2 meters of LDPE extrusion;
N diffusers;
N/2 high-rate diffusing membranes,
N/2 low-rate diffusing membranes,
Lower manifold sections sized for each linear run of columns
Upper manifold sections sized for each linear run of columns.

2) Attach upper U couplers to upper manifold, hang from superstructure and interconnect manifolds.

3) Put rigid cylindrical terminations on 2-meter lengths of LDPE.

4) Connect column's upper ends to U couplers.

5) Assemble diffusers and attach to lower manifold sections,

6) Attach lower U couplers to lower ends of columns.

7) Interconnect manifold sections with elbows and t joints as needed to the configuration.

8) Attach and configure input and output ports

9) Attach and configure valves sensors and control systems.

10) Attach sources of inputs and sinks of outputs as appropriate for desired production.

### General Operation of Unit:

a. Assemble and connect unit
b. Fill with sterile media
c. Sterilize unit and initial medium in situ
d. Initiate flow
e. Add inoculums
f. Operate unit in partial shade until predetermined density is achieved
g. Operate unit in increasing amount of light.
h. Temperature controlled with a sensor activating a cooling system.
i. The system will run as the culture density increases
j. pH will be controlled by injection of CO₂.
k. Harvest either by increments of by batch
l. When required by negative state of culture:
   Empty, disassemble, dispose of plastic bag material,
   Detach U couplers and diffusers from manifold.
   Clean U couplers and diffusers in commercial dishwasher or the like.

### Variations

The columns might be constructed from a rigid material; this might allow a row to be tilted to more effectively capture sunlight. The upper and lower U couplers might be of different designs. Input and output ports can have many various know to those skilled in the art. Columns might be of a different diameter 295 and/or height. Rather than continuously recalculate, a system according to these teachings could be configured as a continuous system with an input and an output.

### Another Version

Consistent with the teachings of this invention, the several parallel columns might be constructed by 2 sheets of plastic bonded to each other at parallel lines with a consistent spacing. Held up with the “columns” each extending along a “plumb line” orientation, this design could produce a row of effective columns. A portion of a U coupler function could also be constituted by bonding the sheets 305 together along short lines perpendicular to the lines creating the columns. Suitable diffusers along the lower edge would be configured and attached.

Those skilled in the art will understand the materials and techniques required to construct and operate a bioreactor consistent with the teachings herein without undue experimentation.

### REFERENCE LIST

1) Up column
2) Upper U coupler
3) Down column
4) Lower U coupler
5) Diffuser body
6) Gas manifold
7) Harvesting port
8) Down column water level
9) Alternate upper U coupler
10) Diffuser having a flow adjusting valve
11) Structural support for making rigid connection between adjacent U-couplers
12) Rigid Collar to terminate Low Density Poly Ethylene column
18) Diffuser outer body, affixed to manifold
19) A & 19B auxiliary port on lower U coupler
20) valve for media intake port
21) media intake port
22) inoculum intake port
23) aux port for gas out on alternate upper U coupler
24) up column water level
25) valve, innoculum port
26) valve, harvest port
27) valve, gas inlet on lower manifold
28) upper manifold
30) bubble
31) elastomeric diffuser membrane—300 larger holes for up column
32) elastomeric diffuser membrane—300 larger holes for up column
33) elastomeric diffuser membrane—300 smaller holes for down column

What is claimed:

1. A photobioreactor comprising: a plurality of generally parallel, vertical columns, said columns linked in a continuous serpentine for flow of a liquid, a gas diffuser or injector proximate to the lower extremity of a plurality of non-adjacent columns (called up columns), a gas diffuser of a less rate proximate to the lower extremity of one or more of the remaining columns immediately adjacent to and down flow of an up column.

2. A photobioreactor comprising: a plurality of generally parallel, vertical columns, said columns linked in a continuous serpentine for flow of a liquid, a gas diffuser or injector proximate to the lower extremity of a plurality of non-adjacent columns (called up columns), a mechanism for mixing cells in the down tubes comprising one or more of bubbles, vibration, magnetic stirring and mechanical stirring.

3. A kit of parts to construct a photobioreactor as described in the claim 1.
